(19) United States
(12) Patent Application Publication
(10) Pub. No.: US 2026/0061135 A1
(43) Pub. Date: Mar. 5, 2026

(54) DOSING APPARATUS FOR PARENTERAL DELIVERY OF FLUIDS

(71) Applicant: SHL Medical AG, Zug (CH)

(72) Inventors: Florence Olivia Stevenson, Baltimore, MD (US); Mariano Mumpower, Baltimore, MD (US); Benjamin Richard Lane, Baltimore, MD (US); Joshua Thomas Hartl, Baltimore, MD (US); Stephen Allison Porter, Baltimore, MD (US); Martin Michael Coyne, III, Boonton, NJ (US); Christoph Egloff, Zug (CH); Christopher James Franzese, Boonton, NJ (US)

(21) Appl. No.: 19/107,173

(22) PCT Filed: Sep. 1, 2023
(86) PCT No.: PCT/EP2023/074082

§ 371 Date: Feb. 27, 2025

Related U.S. Application Data
(60) Provisional application No. 63/403,921, filed on Sep. 6, 2022.

(30) Foreign Application Priority Data
Nov. 14, 2022 (EP) 22207248.0

(51) Int. Cl. A61M 5/315; A61M 5/142; A61M 5/20

(52) U.S. Cl. CPC A61M 5/31528; A61M 5/1422; A61M 5/2066; A61M 5/31596

(57) ABSTRACT

The present disclosure provides a dosing apparatus for parenteral delivery of at least two fluids, the dosing apparatus comprising a dosing actuating unit configured to actuate delivery of one of the at least two fluids, and at least two dosing delivery devices, each dosing delivery device comprising a chamber extending along an axis A comprising at least one intake opening and at least one outlet opening, a first and a second piston, wherein said first and second pistons are displaceable along the axis A within the chamber, wherein the chambers of the at least two dosing delivery devices are configured to each hold a fluid, such that a rotational movement of the dosing actuating unit delivers the fluids.

### CROSS-REFERENCE TO RELATED APPLICATIONS

The present application is a U.S. National Phase Application pursuant to 35 U.S.C. § 371 of International Application No. PCT/EP2023/074082 filed Sep. 1, 2023, which claims priority to (i) U.S. Provisional Application No. 63/403,921 filed Sep. 6, 2022, and (ii) EP Patent Application No. 22207248.0 filed Nov. 14, 2022. The entire disclosure contents of these applications are herewith incorporated by reference into the present application.

### TECHNICAL FIELD

The present disclosure relates to the technical field of administration of therapeutic fluids. In particular, the present disclosure relates to the technical field of dosing apparatuses for parenteral delivery of two or more fluids.

### BACKGROUND

In the administration of liquid formulations of pharmaceutical agents, it is in most cases necessary to deliver well defined volumes. Often drugs must here be injected into the body of a patient. For the parenteral injection, hypoderminc syringes, drug pens or motor driven systems are employed. In particular in the case of preparations which have to be administered over a lengthy period and/or according to a precisely specified schedule, syringes and pens are increasingly being replaced by motor driven systems. Many motor driven systems exist to deliver medicaments, as in the case of parenteral delivery. For instance, peristaltic drives or piston pumps are common in the art.

Depending on the design, the motor driven systems are suitable for being attached over lenghty period to the body of a patient and for administrating a preparation continuously and/or according to an individually specified schedule. Systems of this type typically comprise a container for the liquid formulation, and a conveying device, which conveys the drug to a port of the device or to an injection system.

However, some therapeutic treatments require multiple medications to be administered to the patient. Multiple medications must remain fluidically separate to prevent mixing of medications and potential compatibility issues between medications and/or medication formulation components (i.e., excipients) that may cause unintended or undesirable effects.

Different medications used with the system may each have different volumes and/or viscosities. For instance, some medications may be fixed doses, while others may be variable doses that are patient specific. In other instances, there may be numerous medications, while in others, there may be fewer medications, complicating efficient manufacturing of a delivery device.

While a single medication device need not be concerned with this, this no longer holds true when the pump is intended to deliver two or more drug products.

Accordingly, one substantial limitation is the number of actuators required to drive each pump scales linearly. Prior art efforts to use a single motor, as in the case of multi-channel peristaltic pumps, have significant mechanical complexity. Both limitations contribute to high cost, thus limiting broad applicability.

Thus, there is a need for improved systems and apparatus for administration that provide new solutions to the problems seen in the prior art.

### SUMMARY

It is realized as a part of the present disclosure that there are limitations in prior solutions configured to deliver multiple medicaments, and in particular multi-medication regimens. These limitations may imply an increased complexibility in the delivery of medicaments, potentially causing a discomfort with a receiving patient. Furthermore, an increased complexity may also demand more from the professional health carers administering the medicaments, for example in terms of working hours, training and specific knowledge about each medicament to be delivered.

It is further realized as a part of the present disclosure that parenteral delivery of multiple medicaments may cause undesirable effects if the medicaments are not kept fluidically separate. Undesirable effect for example include loss of potency, efficiancy or aggregation. This may further add to the complexity of multiple medicament delivery, along with posing a safety risk for the receiving patient.

Therefore, the inventors have come to the realization that there is a need for an apparatus for administration that allows flexible delivery of multiple medications that does not require a priori knowledge of the number, volume or viscosity of the medicaments, and that may be flexibly configured to deliver one or more medicaments in a desired sequence, governed by a prescribed medication regimen.

The present disclosure therefore provides a dosing apparatus for parenteral delivery of at least two fluids, a medicament delivery device comprising the dosing apparatus and a method for parenteral delivery of two or more fluids in the independent claims, that can mitigate the above-mentioned drawbacks. Preferred embodiments are defined in the dependent claims.

Hence, according to a first aspect of the present disclosure, there is provided a dosing apparatus for parenteral delivery of at least two fluids, the dosing apparatus comprising: a dosing actuating unit comprising: a cam element rotatably arranged around a rotational axis; and a cam profile extending from the cam element. The cam element is configured to be connected to a drive unit and to be rotated by the drive unit around the rotational axis. The dosing apparatus further comprises at least two dosing delivery devices, each dosing delivery device comprising: a chamber extending along an axis and a first piston movably arranged along the axis within the chamber. The chamber is configured to accommodate a fluid. The chamber comprises an outlet opening for delivery of the accommodated fluid. The first piston comprises a first cam follower configured to releasably engage with the cam profile of the dosing actuating unit; wherein an interface formed between the cam profile and the first cam follower is helical such that a rotational movement of the cam element is converted into an axial movement of the first piston along the axis of the chamber.

A diameter of the cam element together with the shape of the cam profile may determine the number of dosing delivery devices arranged around the cam element. An increased diameter of the cam element may increase the number of dosing delivery devices that may be arranged around the cam element. A decreased diameter of the cam element may decrease the number of dosing delivery devices that may be arranged around the cam element. Since each dosing delivery device may deliver a fluid, the number of fluids delivered may increase with the number of dosing delivery devices arranged around the cam element. The size of the dosing apparatus may be adapted to the number of fluids to be delivered. However, all sizes of dosing apparatus may be used for the delivery of the at least two fluids.

The cam element may have a length L. The dosing delivery device may have the same length as the cam element. The dosing delivery device may also have a longer or shorter length than the cam element.

Preferably, according to one embodiment, the cam profile comprises a plurality of profile segments arranged on the cam element and distributed along the rotational axis of the cam element. This embodiment may allow for individual control of the at least two dosing delivery devices with one cam element. For example, one cam profile segment may be engaged with the first piston of one of the at least two dosing delivery devices, while another cam profile segment may be engaged with the first piston of another one of the at least two dosing delivery devices.

Preferably, according to one embodiment, the at least two dosing delivery devices are identical.

Preferably, according to one embodiment, the chambers of the at least two dosing delivery devices have different dimensions to one another. For example, one chamber of the at least two dosing delivery devices has a different diameter and/or different length (measured along the axis of the chamber) to another chamber of the least two dosing delivery devices. Therefore, the at least two dosing delivery devices can deliver different amounts of the same or different accommodated fluids.

In the second aspect of the present disclosure, there is provided a medicament delivery device comprising the dosing apparatus, wherein the medicament delivery device comprises: a drive unit operably connected to the cam element; a control unit configured to control the drive unit to activate one of the at least two dosing delivery devices to selectively deliver one of the at least two fluids, or deliver the at least two fluids.

Preferably, according to one embodiment, the drive unit comprises a stepper motor.

Preferably, according to one embodiment, the drive unit comprises a gear set.

Preferably, according to one embodiment, the drive unit comprises two stepper motors connected to one another via the gear set.

The control unit may for example control the rotating speed or direction of the cam element, by controlling operation of the drive unit. In one exemplifying embodiment, the drive unit is one or more stepper motors. Thus, by controlling the stepper motor, the control unit may control operation of the dosing apparatus.

Further, the drive unit may be arranged on the rotational axis of the cam element to indirectly drive the cam element through the rotational axis of the cam element. The control unit may be any type of computer, embedded system or input-output device suitable for controlling a drive unit such as a stepper motor. The present embodiment is advantageous in that the control unit may provide controllability of the apparatus. By controlling a speed and a direction of the cam element, the dosing speed can be controlled, which allows for different volumes and viscosities of fluids to be delivered. This may be advantageous in that it may further increase the flexibility and usability of the apparatus. It will be appreciated that embodiments of the drive unit other than a stepper motor are possible.

In the third aspect of the present disclosure, there is provided a method for parenteral delivery of two or more fluids using the dosing apparatus according to the first aspect of the disclosure. The method comprises receiving an indication to start a delivery process of the at least two fluids in a predefined sequence. The indication may for example be given by a professional health carer or a patient to start the treatment. The method further comprises causing the delivery actuating unit to engage with the first dosing delivery device in the sequence. This may include controlling the drive unit to rotate the cam element such that it may engage with the dosing delivery device. The method further comprises causing the cam element to rotate such that the fluid flows into an intake opening of the chamber from the dosing delivery device. The method further comprises causing the cam element to rotate such that the fluid flows out from an outlet opening of the chamber. The method further comprises causing the delivery actuating unit to engage with the next dosing delivery device in the sequence.

By the term “fluid” is herein meant a liquid medicament suitable for parenteral delivery to a patient.

By the term “cam element” is herein meant a rotating or sliding piece, for example with a substantially cylindrical shape, used to transform rotary motion into linear motion or vice versa.

By the term “cam profile” is herein meant a protrusion or a groove arranged on the surface of the cam element, to drive a follower. The follower is a term used to describe the device which the cam element is in contact with to transform its rotary motion into a linear motion of the follower.

Thus, the apparatus according to the present disclosure allows for administration of multiple medicaments. Further, the apparatus may be used to deliver multiple medicaments without knowledge of the number, volume or viscosity of the medicaments beforehand. This is advantageous since it increases the flexibility of the apparatus. A further advantage from this may be that the apparatus can be used in a wide range of applications, resulting in an increased usability. Further preferred embodiments of all aspects of the invention are presented as follows.

Preferably, according to one embodiment, each dosing delivery device of the at least two dosing delivery devices comprises a second piston movably arranged along the axis within the chamber; wherein the second piston comprises a second cam follower configured to releasably engaged with the cam profile of the dosing actuating unit.

Preferably, according to one embodiment, fluids are prefilled within the chambers.

Alternatively, according to one embodiment, the chamber comprises an intake opening open in a direction transverse to the axis; wherein the intake opening is configured to be fluidly connected to a container containing a fluid; wherein the outlet opening is open in the direction transverse to the axis.

The container may be a flexible bag filled with a medicament, for example.

An action that the fluid flows from the container via the intake opening into the chamber is referred as a suction action in the description. An action that the fluid is expelled from the outlet opening out of the chamber is referred as a pumping action in the description.

The first piston, second piston and the chamber cam delimit a variable fluid volume. The first and second pistons are arranged in the chamber along the axis. The length of the first and second pistons may be smaller than a length of the chamber. Further, a total length of the first piston together with the second piston may be smaller than a length of the chamber, such that there is room for a fluid. In one example, the chamber and the pistons may have a substantially cylindrical shape. The diameter of the first and second pistons may be smaller than a diameter of the chamber. Thus, the first and second pistons may not fill the space delimited by the chamber. Therefore, a variable fluid volume may fill at least a portion of the remaining space within the chamber. Thus, different fluid volumes may be used with the same dosing delivery device. This is advantageous since different medicaments may require different dose sizes.

The at least one intake opening and the at least one outlet opening in the chamber may be arranged on a surface of the chamber. The intake and outlet openings may be arranged adjacent to each other on the surface of the chamber. The intake and outlet opening(s) may further be arranged adjacent to each other in paralell with the axis of the chamber. The at least one outlet opening may be connected to a fluid path leading the fluid to a receiving patient. The fluid path connected to the at least one outlet opening may for example be a tube connected to the at least one outlet opening in one end, and connected to a receiving patient in the other end. It will be appreciated that other embodiments of connecting a fluid to the dosing apparatus are possible, and outside the scope for the present disclosure.

In a first position, where an acutation of the first and second pistons has not yet occurred, the first and second pistons may cover an interior surface of the chamber where the intake and outlet openings are positioned. Further, upon acutation of the first or second piston, a axial movement of the first or second piston may uncover the intake or outlet opening, such that a fluid may be allowed to flow into the chamber. Further, upon axial movement of the other one of the first or second pistons, the first or second piston may uncover the at least one outlet opening, such that the fluid may be allowed to flow out from the chamber, and be delivered to a patient. The first and second pistons may move alone or together. In the event where the first and second pistons move together, the fluid volume inside the chamber may be moved along A.

Furthermore, a axial movement of the first and/or second pistons may create a negative pressure inside the chamber, which may cause the fluid to be drawn into the chamber through the at least one intake opening. The direction of the axial movement may be determined by the direction of rotation of the cam element.

Preferably, according to one embodiment, the dosing delivery device is a positive displacement pump.

Preferably, according to one embodiment, the chamber together with the first and second pistons, and the fluid volume may act as a positive displacement pump.

Preferably, according to one embodiment, the positive displacement pumps have substantially unequal displacement volumes per stroke.

Preferably, according to one embodiment, the chambers of the at least two dosing delivery devices have unequal diameters.

The fluid may be arranged between the first and second piston along within the chamber. Upon axial movement of the first and/or second piston, the fluid may therefore move along A within the chamber. Since the chamber further comrpises at least one intake opening and at least one outlet opening, the fluid may be allowed to enter and exit the chamber.

Preferably, according to one embodiment, the at least two dosing delivery devices are radially arranged around a circumference of the cam element.

Preferably, according to one embodiment, the axis of the chamber of each dosing delivery device is parallel to the rotational axis of the cam element.

Preferably, according to one embodiment, the distance between one of the chamber of the at least two dosing delivery devices and the cam element in the direction transverse to the axis of the chamber are equal to another one of the chamber of the at least two dosing delivery devices and the cam element in the direction transverse to the axis of the chamber.

The dosing apparatus may thus deliver two or more fluids during one operation. In an exemplifying embodiment, there are four dosing delivery devices radially arranged around a circumference of the cam element. In another exemplifying embodiment, there are three dosing delivery devices arranged around a circumference of the cam element.

Preferably, according to one embodiment, the distance between one of the chamber of the at least two dosing delivery devices and the cam element in the direction transverse to the axis of the chamber are unequal to another one of the chamber of the at least two dosing delivery devices and the cam element in the direction transverse to the axis of the chamber.

Preferably, according to one embodiment, wherein the cam profile is at least partially encircling a circumference of the cam element.

The cam profile may for example extend around a quarter of the diameter of the cam element. In another example, the cam profile may extend around the entirety of the diameter of the cam element. The length of the cam profile may determine an operation of the dosing apparatus.

Preferably, according to one embodiment, the cam profile helically extends at least partially around the cam element. The helical extension of the cam profile around the cam element may define the axial movement of the first and second pistons. Further, a combination of the length of the cam profile and the helical extension may determine a length of the axial movement of the first and second pistons. The direction of the axial movement of the first and second pistons may be determined by the helical extension of the cam profile. For example, if the helical extension covers the entirety of the periphery of the cam element, the first and/or second piston may move back and forth during one revolution of the cam element. Thus, the cam element may actuate a reciprocating motion with the first and second pistons. This may be advantageous in that the first and second pistons may deliver a portion of the fluid, and then return to an initial position. From the initial position, the operation may be repeated until all fluid is delivered. The fluid to be delivered may have a greater volume than what fits inside the chamber. Therefore, the container holding the fluid connected to the at least one intake opening may be emptied first after a number of cycles have been performed by the dosing apparatus.

Preferably, according to one embodiment, the helical extension of the cam profile covers a portion of the periphery of the cam element. The cam element may thus rotate back and forth to deliver the fluid. An axial movement of one of the pistons may be defined as one stroke. Thus, the shape and length of the cam profile may determine a stroke size of the first and second pistons. This may be advantageous in that the dosing apparatus may operate in cycles, such that various fluid volumes may be delivered. This may be advantageous in that the dosing apparatus may deliver fluids independently from their respective fluid volume.

Preferably, according to one embodiment, the first cam follower is a rib protruding from the first piston in a direction transverse to the axis of the chamber.

Preferably, according to one embodiment, the chamber comprises at least a first piston opening through which the first cam follower protrudes; and wherein the first piston opening is open in the direction transverse to the axis of the chamber.

Preferably, according to one embodiment, the second cam follower of the second piston is a rib protruding from the second piston in a direction transverse to the axis of the chamber.

Preferably, according to one embodiment, the chamber comprises at least a second piston opening through which the second cam follower protrudes; and wherein the second piston opening is open in the direction transverse to the axis of the chamber.

Preferably, according to one embodiment, the first and/or second piston openings are slots.

The ribs protruding from the first and/or second pistons are configured to releasably engage with the cam profile extending from the cam element, as mentioned above. The first and second cam followers may be slidably engaged with the cam profile, such that when the cam element rotates, the first and second cam followers move along the cam profile around the cam element. The shape of the cam profile around the cam element may thus determine the axial movements of the first and second pistons.

Alternatively, the cam profile comprises a rib, and the first and/or the second cam followers is a recess.

Preferably, according to one embodiment, the cam element comprises a first cam member and a second cam member, wherein said first and second cam members are spaced apart from each other along the axis of the cam element.

Preferably, according to one embodiment, the cam profile comprises a first segment arranged on the first cam member, and a second segment arranged on the second cam member. The cam profile segments may thus be arranged to engage with one of the first and second pistons each. This means that each cam segment may control an axial movement of each piston. In a preferred embodiment, the drive unit of the medicament delivery device comprises a first cam drive and a second cam drive. For example, the first and the second cam drives can be two stepper motors, two gear sets operably connected to one stepper motor or two gear sets operably connected to one stepper motor via a gear set. The first cam drive is configured to actuate a rotational movement of the first cam member, and the second cam drive is configured to actuate a rotational movement of the second cam member. This may further increase the controllability of the dosing apparatus, since the first and second cam members may be controlled individually by a first or second cam drive. Further, according to another embodiment, the first cam member and the second cam member are arranged to rotate independently from each other. The first and second cam members may thus rotate simultaneously in different directions. Each cam drive may independently actuate a movement of each cam member. Thus, there may be a cam drive for controlling the suction action of one of the pistons, i.e. drawing in fluid from the at least one intake opening of the chamber. Further, there may be a cam drive controlling the pumping action of one of the first and the second pistons of the dosing delivery device, i.e. allowing fluid to exit through the at least one outlet opening of the chamber.

Preferably, according to one embodiment of the second aspect of the invention, the medicament delivery device comprises a sensor electrically connected to the control unit; wherein the sensor is configured to detect the position of the cam profile.

Preferably, according to one embodiment of the second aspect of the invention, the sensor is arranged on the cam element or arranged within the drive unit.

Preferably, according to one embodiment of the second aspect of the invention, the sensor is at least one of an optical sensor, a magnetic sensor, or an encoder strip.

Preferably, according to one embodiment of the second aspect of the invention, the control unit is configured to, based on information from the sensor regarding the position of the cam profile, control the drive unit such that the cam element is rotated and engaged with the dosing delivery device for the selected fluid.

The control unit may further control an operation of the drive unit to activate at least one of the dosing delivery devices. The control unit may control a position of the cam profile on the cam element by rotation. The at least two dosing delivery devices may be arranged in a predefined initial position adjacent the cam element. The control unit may actuate a rotation of the cam element such that a start or an end of the cam profile may correspond to that initial position. According to the predefined sequence, one dosing delivery device at a time may then be engaged with the cam profile, such that the delivery of the fluid may start. The control unit may then control the cam element to return to the initial position after delivery of the first fluid, and engage with the next dosing delivery device in the predefined sequence.

The control unit may thus control an operation of the drive unit such that the at least two fluids are delivered in a predefined sequence. The dosing apparatus may be controlled to perform a number of “cycles” until the desired doses of the fluids in the sequence are delivered. In this embodiment, the number of cycles to is preset. This may be advantageous in that a number of fluids may be delivered by the dosing apparatus, regardless of the size of the dose or viscosity of the fluids. Further, a predefined sequence of the fluids to be delivered may be set by a professional health carer, or according to a predefined medication regimen.

The dosing delivery device may be disengaged from the dosing actuating unit by controlling a position of the cam profile to disengage from the first and/or the second cam followers.

Furthermore, the control unit can control the dosing apparatus to continue to deliver fluids until a source of fluids, e.g., the container as mentioned above, is fully emptied. In this embodiment, “End of Dose” could be detected by the sensor, e.g., by monitoring the drive unit, like monitoring the motor torque or current during the suction action as it would be harder to move the piston through the intake opening of the chamber when the bag is empty.

This may be advantageous in that an operation of the dosing apparatus may be automatic, not requiring any manual control by a professional health carer, or similar.

Preferably, according to one embodiment of the second aspect of the invention, the control unit controls an operation of the drive unit such that the at least two fluids are delivered in a predefined sequence.

Preferably, according to one embodiment of the second aspect of the invention, the control unit is configured to, based on a trigger input regarding the need for an emergency medication fluid delivery, adapt an operation of the drive unit such that, regardless of the predefined sequence, the emergency medication fluid is delivered next in order.

A professional health carer may identify an emergency medicament in the predefined sequence of fluids. Upon a triggering signal from a user, for example a patient or the professional health carer, the control unit may control an operation of the cam elements such that the dosing delivery devices prior to the emergency medicament in the sequence may be skipped. For example, the control unit may rotate the cam element such that the first and second pistons of the engaged dosing delivery device may move simultaneously to not deliver any fluid to the patient. Further, the cam element may engage with the next dosing delivery device in the sequence, and perform the same action, i.e. rotating the first and second pistons simultaneously such that no (or substantially no) fluid is delivered. When the emergency medicament is next in the sequence, the operation of the dosing apparatus may return to the “normal” operation, delivering the fluid to the receiving patient. One advantage of this embodiment is that the “skipping” of stations delivers no volume.

The present embodiment may be advantageous in that a patient may receive an emergency medicament without having to wait for it to be administered in the predefined sequence.

Preferably, according to one embodiment of the third aspect of the invention, the method for parenteral delivery of two or more fluids using the dosing apparatus further comprises receiving an indication to start a delivery process of an emergency fluid in the predefined sequence, causing the delivery actuating unit to skip all fluids in the sequence except for the emergency fluid.

Preferably, according to one embodiment of the second aspect of the invention, wherein the control unit is configured to control the drive unit to rotate the cam element, such that upon rotational movement of the cam element, the cam element actuates a movement of the first piston along A, causing a fluid to flow in through the one or more intake openings of the chamber, and a movement of the second piston along A, causing the fluid to flow out through the one or more outlet openings of the chamber. As previously mentioned, the first and second pistons are configured to be releasably engaged with the cam profile.

Preferably, according to one embodiment of the second aspect of the invention, the drive unit is a stepper motor.

Preferably, according to one embodiment of the second aspect of the invention, the medicament delivery device comprises an indexing member configured to be releasably engaged with a dosing delivery device at a predefined starting position, such that upon activation of a delivery of a fluid from the at least two fluids, the dosing actuation unit engages with the dosing delivery device containing the fluid in the starting position, whereby the dosing delivery device is released from the indexing member.

Preferably, according to one embodiment of the second aspect of the invention, the medicament delivery device further comprises a frame configured to at least partly enclose the dosing actuating unit and the at least two dosing delivery devices.

The frame may provide protection for the cam element and the dosing delivery devices. Further, the dosing apparatus may be easier to transport when enclosed by a frame. The frame may for example be a type of housing. The housing may be made of a transparent, opaque, or partially opaque material, such as a type of hard plastic, and may optionally be configured to allow inspection of the apparatus or the medication(s) therein.

Preferably, according to one embodiment of the second aspect of the invention, the indexing member is fixed to the frame, and adjacent to one of the at least two delivery devices along the axis of the chamber.

Preferably, according to one embodiment of the second aspect of the invention, the indexing member is an elastic member.

Preferably, according to one embodiment of the second aspect of the invention, the elastic member is a compression spring or an elastic cushion.

The indexing member may be arranged adjacent a dosing delivery device along the axis of the chamber. For example, a compression spring may be loaded while engaged with a dosing delivery device in a starting position. The cam follower may then be maintained in a position where the cam profile may engage with the cam follower. Upon engagement with the cam profile, the rotation of the cam element may overcome a loaded force from the compression spring, such that the dosing delivery device may follow the cam profile during rotation of the cam element. Upon engagement with the cam profile, the loaded compression spring may no longer need to support the dosing delivery device, upon which it may return to its unloaded position. The present embodiment may be advantageous in that the dosing delivery device(s) may be installed in the dosing apparatus such that they are ready to be engaged with the cam element. This may further automate the control of the dosing apparatus. Other methods than a compression spring may obviously use to provide such a loaded force.

Preferably, according to one embodiment of the second aspect of the invention, the indexing member is configured to break upon start of the engagement or releasement between the cam profile and first and/or cam followers of a dosing delivery device, such that engagement with a further dosing delivery device is prevented.

This may for example be beneficial in a case where it is desired to ensure that a fluid is delivered only once. The indexing member may for example be a piece of plastic, weak enough to not withstand the rotational force from the cam element upon engagement with the dosing delivery device.

It should be noted that the indexing member may not be needed if the seals between the first and/or the second pistons and chamber provide sufficient friction to maintain a starting position prior to cam engagement.

According to an embodiment of the present disclosure, the indexing member is fixed to the frame, and adjacent to one of the at least two delivery devices along A.

Preferably, according to one embodiment of the second aspect of the invention, when the cam element comprises the first and the second cam members, the control unit is configured to synchronize the movement of the first cam member and the second cam member through the first and second cam drives. The suction action and the pumping action of the dosing delivery devices may thus be synchronized by the first and second cam drives. Further, according to an exemplifying embodiment of the present disclosure, the first cam drive and the second cam drive are connected to each other by gear train or a splined connection. The connection between the first and second cam drives may thus enable the synchronization of the first and second cam members.

Preferably, according to one embodiment of the second aspect of the invention, the medicament delivery device is an injection device, e.g., an autoinjector or a manually injector, an infusion device, an inhalation device, or a medical sprayer.

Preferably, according to one embodiment of the second aspect of the invention, the medicament delivery device comprises a user wearable feature for being worn by a patient, so that the patient mobility can be improved during the delivery of the fluid.

Preferably, according to one embodiment of the second aspect of the invention, the user wearable feature can be a shoulder strip, a wrist band, a neck strip, a wariest belt and/or a belt clip.

Preferably, according to one embodiment of the second aspect of the invention, the medicament delivery device is configured to connect with a delivery member to deliver a fluid to a patient. The delivery member can be an injection needle or a spray nozzle or a soft cannular.

The medicament delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis, allergies, oncology, and immune deficiencies (e.g., primary immunodeficiency). Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies.

Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives.

Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), immunoglobulins (primary immune deficiencies), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier.

Pharmaceutical formulations comprising a drug as listed herein (or a pharmaceutically acceptable salt of the drug) may include one or more other active ingredients, or may be the only active ingredient present. Pharmaceutical formulations may also include separately administered or co-formulated dispersion enhancers, such as hyaluronidase.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

It is to be understood that the above embodiments are described using one dosing delivery apparatus as reference. This is to provide a view of how the relation between a dosing delivery device and the cam element may look. It will be appreciated, however, that this does not delimit the dosing apparatus to comprise, or be used with, only one dosing delivery device. The relation between the dosing delivery device and the cam element remains the same with the increasing number of dosing delivery devices arranged on the cam element.

### BRIEF DESCRIPTION OF THE DRAWINGS

One or more embodiments will be described, by way of example only, and with reference to the following figures, in which:

FIGS. 1A-1C schematically illustrate a dosing apparatus according to an embodiment of the present disclosure.

FIGS. 2A-2D illustrate a dosing delivery device according to an embodiment of the present disclosure.

FIGS. 3A-3E illustrate a delivery cycle of a dosing apparatus according to an embodiment of the present disclosure.

FIG. 4 illustrates a dosing apparatus comprising a frame and an elastic member according to an embodiment of the present disclosure.

FIGS. 5A-5B illustrate possible embodiments of the dosing actuating unit.

### DETAILED DESCRIPTION

The present disclosure is described in the following way by way of a number of illustrative examples. It will be appreciated that these examples are provided for illustration and explanation only and are not intended to be limiting on the scope of the disclosure.

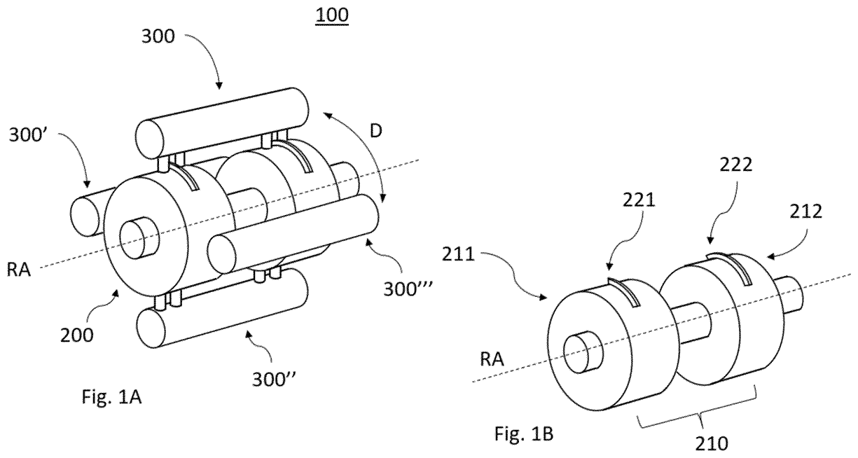

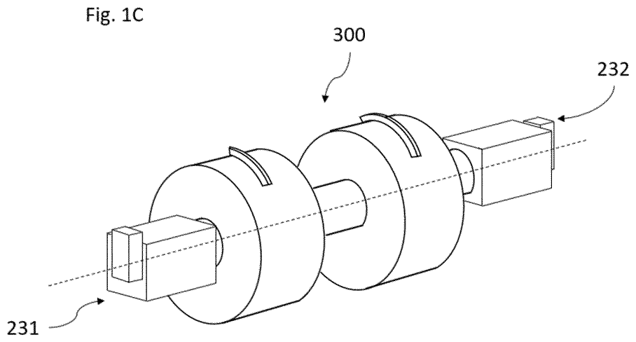

FIG. 1A illustrates a dosing apparatus 100 according to an embodiment of the present disclosure. The dosing apparatus 100 comprises a dosing actuating unit 200 arranged along an rotational axis RA. Around the dosing actuating unit 200 there are four dosing delivery devices 300, 300′, 300″, 300′″ arranged. The dosing delivery devices 300, 300′, 300″, 300′″ are arranged with a distance D to each other. Further, each of the dosing delivery devices 300, 300′, 300″, 300′″ comprises a chamber 310 have a substantially cylindrical shape extending along an axis A. The axis A of the chamber 310 and the rotational axis RA are parallel, such that the dosing delivery devices 300, 300′, 300″, 300′″ are radially arranged around the dosing actuating unit 200.

FIG. 1B illustrates a dosing actuating unit 200, arranged along the rotational axis RA, in more detail. The dosing actuating unit 200 comprises a cam member 210. In a preferred example, the cam member 210 comprises a first cam member 211, and a second cam member 212. The first 211 and second 212 cam members are arranged with a distance to each other along the axis RA. The first 211 and second 212 cam members have a substantially cylindrical shape. Further, on the first cam member 211 there is a first cam profile segment 221. Similarly, on the second cam member 212 there is a second cam profile segment 222 arranged. The first 221 and the second 222 cam profile segments extend helically around a surface of the first 211 and second 222 cam members. Further, the first 221 and second 222 cam profile segments are protruding radially from the axis RA from a surface of the first 211 and second 222 cam members.

FIG. 1C illustrates the dosing apparatus 100 with a first 231 drive of a medicament delivery device and a second 232 cam drive of the medicament delivery device arranged on the rotational axis RA (not shown in this Figure). The first 231 and second 232 cam drives may be stepper motors. The first 231 and second 232 cam drives are arranged to indirectly actuate a rotational movement of the first 211 and second 121 cam members, via the rotational axis RA.

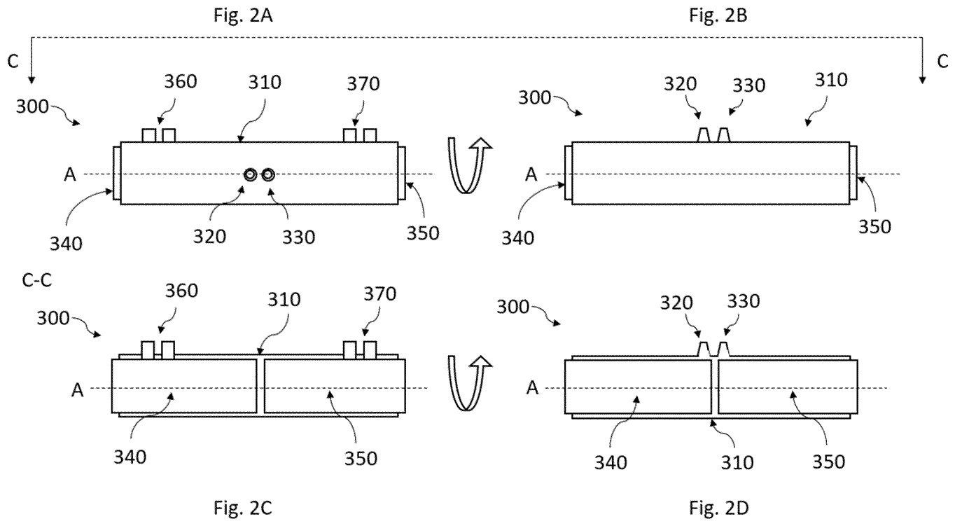

FIGS. 2A-2D illustrate a dosing delivery device 300 according to an embodiment of the present disclosure. The dosing delivery device 300 comprises a chamber 310 extending along an axis A, as shown in FIG. 2A. In this example, the chamber 310 partly encloses a first piston 340, and a second piston 350. Further, there is an intake opening 320 and an outlet opening 330 arranged in a body wall of the chamber 310. The intake opening 320 and the outlet opening 330 are arranged along the axis A adjacent to each other. In a preferred example, the intake opening 320 is open in a direction transverse to the axis A of the chamber 310, and the outlet opening 330 is open in the direction transverse to the axis A of the chamber 310. In a preferred example, the intake opening 320 and the outlet opening are open in the same direction, as shown in FIGS. 2A-D. Furthermore, the first piston 340 comprises a first cam follower 360 and the second piston 350 comprises a second cam follower 370. The first 360 and second 370 cam followers are arranged towards the opposite ends of the dosing delivery device 300 along the axis A. In a preferred example, the first 360 and second 370 cam follower are a pair of protruding ribs, or forks (formed by multiple ribs), prodtruding radially from the axis A of the chamber 310. Further, the first 360 and second 370 cam followers are arranged on the first 340 and second 350 pistons, respectively. Further, the intake opening 320 and the outlet opening 330 are arranged with an approximately 90°angle to the first 360 and second 370 cam follower around a periphery of the dosing delivery device 300.

This is illustrated in FIG. 2B, wherein the dosing delivery device 300 has been rotated 90°around axis A. From this view, it is seen that the intake opening 320 protrudes from the chamber 310. The shape of the intake opening 320 may be conical in order to mate with a container containing a fluid (not shown in the Figure) or similar. Further, the outlet opening 330 protrudes from the chamber 310. The shape of the outlet opening 330 may be conical in order to mate with a delivery member (not shown in the Figure) for delivery of the fluid to a patient.

FIGS. 2C and 2D illustrate cross-sectional views of the corresponding views as FIGS. 2A and 2B, according to section C. FIG. 2C shows the first piston 340 and the second piston 350, arranged in the chamber 310 along axis A. The first piston 340 is slidably arranged in the chamber 310. As seen in the figure, the first piston 340 is partially enclosed by the chamber 310, such that it is allowed to slide along the axis A of the chamber 310. The second piston 350 is arranged in the chamber 310. In a preferred example, the second piston 350 is lined up with the first piston 340 along the axis A of the chamber 310. The second piston 350 is slidably arranged in the chamber 310. As seen in the figure, the second piston 350 is also partially enclosed by the chamber 310. Further, the first 340 and second 350 pistons are arranged with a space between them. The space allows for a variable fluid volume to be arranged in the chamber 310. In a preferred example, as shown in FIG. 2D, the first and the second piston can be operated with the following steps in the following orders to perform a suction action to causes the fluid flows from the contain into the chamber via the intake opening.
providing the first and the second piston adjacent to one another;
aligning the interface formed between the first and the second piston to with the intake opening of the chamber;
moving the first piston away from the second piston

The reduction of the pressue between the first and the second piston thus causes the fluid flows from the contain into the chamber via the intake opening. When the fluid within the chamber 310 is fluidly connected to the outlet opening, moving the first and the second piston towards one another can thus create the pumping action to cause the the fluid in the chamber to be expellled out of the chamber via the outlet opening.

The first 340 and second 350 pistons have a substantially cylindrical shape, with a diameter smaller than that of the chamber 310. Further, the first 340 and second 350 pistons are arranged in the chamber such that the variable fluid volume is sealed within the space, thus not allowed to leak out from the chamber 310 other than from the intake opening 320 or outlet opening 330. On the first piston 340, the first cam follower 360 are arranged. Similarly, on the second piston 350, the second cam follower 370 are arranged. The chamber 310 has an opening (not shown in the figure) for allowing the first 360 and second 370 cam follower to move along the axis A, such that the first 340 and second 350 pistons may slide along the axis A within the chamber 310.

FIG. 2D shows a cross section of the dosing delivery device 300 from the same view as in FIG. 2B, according to section C. From this view, it can be seen that the intake opening 320 and the outlet opening 330 are covered from an inside of the chamber 310 by the first 340 and second 350 pistons in the present position of said pistons. It may further be seen that the intake opening 320 is in fluid connection with the chamber 310. Similarly, the outlet opening is in fluid connection with the chamber 310.

It should be noted that for the dosing appratus, one dosing delivery device only needs one piston arranged within the chamber. For example, the chamber can be prefilled with a fluid. Therefore, the suction action is not necessary. Thus, the one dosing delivery device may only comprises a chamber with an outlet opening and a first piston to provide the pumping action with the outlet opening of the chamber when the first piston is moved axially along the axis A in the chamber.

The first and the second cam followers are configured to be releasbly engaged with the cam profile of the dosing acutating unit 200. An interface formed between the cam profile and the first cam followers is helical such that a rotational movement of the cam element is converted into an axial movement of the first piston along the axis A of the chamber 310. Similarly, An interface formed between the cam profile and the second cam followers is helical such that a rotational movement of the cam element is converted into an axial movement of the second piston along the axis A of the chamber. For example, the cam profiles 221, 222 as shown in FIG. 1C, are helically extending partially around the cam element 210. Thus, the first and the second cam followers can move along the helical cam profile when the cam member is rotated.

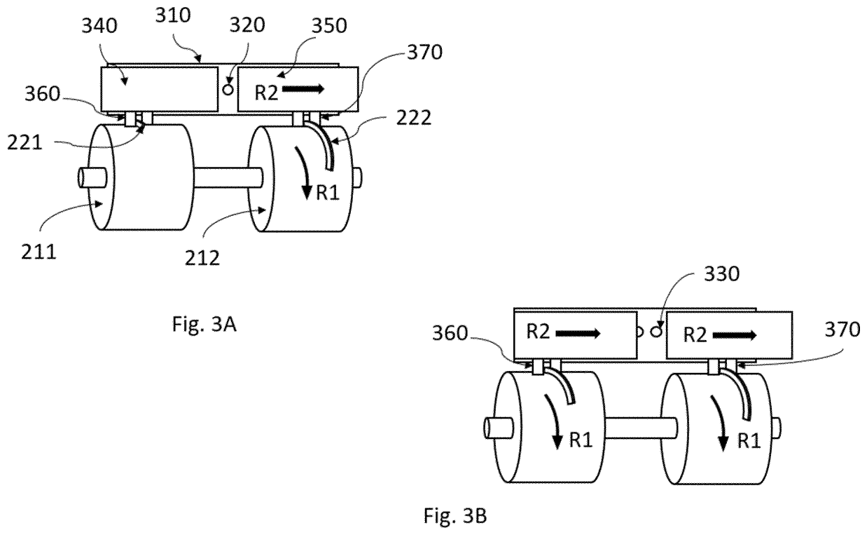

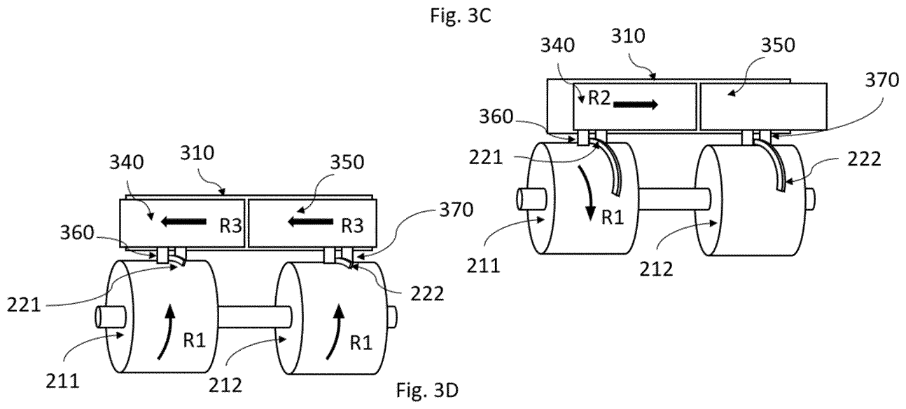

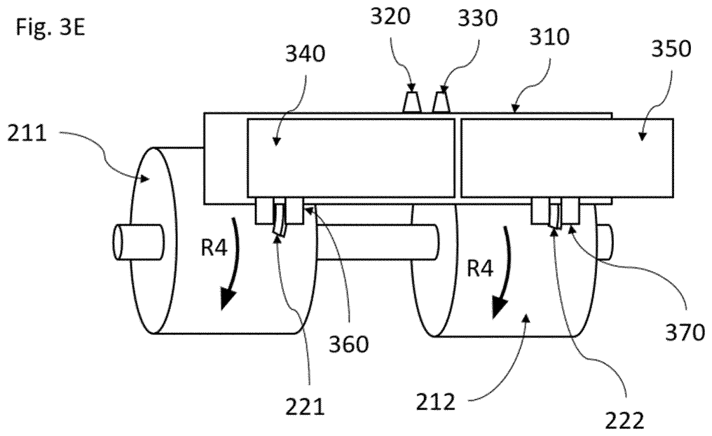

FIGS. 3A-3E show a delivery cycle of a dosing apparatus 100 according to an embodiment of the present disclosure. A cross-sectional view of the dosing delivery device 300 is shown according to section C from FIGS. 2A-D, in order to show the movement of the first 340 and second 350 pistons during delivery of a fluid. The figures illustrate a cycle of one fluid being delivered, to provide an understanding of how the dosing actuating unit 200 works in connection with a dosing delivery device 300. It is appreciated that the illustrated cycle will be the same for a second, third or any number of fluids being delivered.

FIG. 3A depicts a first state of the delivery cycle of a fluid, also referred to as the “suction action”. Prior to this state, the first 211 and second 212 cam members have been rotated by the first 231 and second 232 cam drives (not shown in this Figure) to a starting position wherein the first 360 and second 370 cam follower have engaged with the first 221 and second 222 cam profile segments. The first 221 and second 222 cam profile segments extends along a portion of the periphery of the first 211 and second 212 cam members. To reach the suction state shown in FIG. 3A, the second cam drive 232 have actuated a rotation of the second cam member 212 to actuate a axial movement of the second piston 350. Further, the first cam drive 231 has not actuated a movement of the first cam member 211, why the first cam member 211 is not rotated in this state.

In the current figure, the direction of rotation is illustrated with an arrow R1 in a counterclockwise direction. Note that the direction of the rotation may be the opposite, depending on how the first 221 and second 222 cam profile segments are arranged. The arrows to show a direction of rotation are merely to show an example of how a delivery cycle may be performed.

Further, the second cam profile segment 222 has slid through the second cam follower 370. The extension of the second cam profile segment 222 around the second cam member 212 is helical, thereby a rotation of the second cam member 212 can be converted into an axial movement of the second cam follower 370. The second cam follower 370 are arranged on the second piston 350, thereby an axial movement of the second cam follower 370 turns into an axial movement of the second piston 350. The second piston 350 moves along the axis A of the chamber 310 to uncover an intake opening 320 of the chamber 310 (in a direction illustrated by a horizontal arrow R2 in the figure). Since the chamber may be sealed, an axial movement of the second piston 350 may create a negative pressure inside the chamber 310, causing fluid to be drawn in from the intake opening 320, as mentioned above. The fluid entering the chamber 310 from the intake opening 320 fills up the variable fluid volume space between the first 340 and second 350 pistons.

FIG. 3B depicts a second state of the delivery cycle for a fluid. The first 360 and second 370 cam followers are engaged with the first 221 and second 222 cam profile segments, and the fluid is in the chamber 310. The first cam drive 231 and the second cam drive 232 are synchronized so as to actuate a rotational movement of the first 211 and second 212 cam members simultaneously. The first 340 and second 350 pistons will thus slide axially along the axis A of the chamber 310 simultaneously, in the same direction (illustrated by horizontal arrows R2 in the figure). The fluid inside the chamber 310 will thus be moved axially along the axis A of the chamber 310. Since the first 340 and second 350 pistons move simultaneously, the pressure will remain constant in the chamber 310, such that no fluid will be sucked in or pressed out from the chamber 310. Further, the first piston 340 moves aixally along the axis A of the chamber 310 to cover the intake opening which was uncovered by the second piston 350 in the suction state. The second piston 350 moves further along the axis A of the chamber 310 to uncover the outlet opening 330 in the chamber 310.

FIG. 3C depicts a third state of the delivery cycle for a fluid, also called the “pumping action”. In this state, the first cam drive 231 actuates a rotational movement of the first cam member 211. The second cam member 212 remains in its position from the previous, second, state. Thus, an axial movement of the first piston 340 is actuated (in a direction illustrated by a horizontal arrow R2 in the figure). The first piston 340 moves axially along the axis A of the chamber 310 to remove the space between the first 340 and second 350 pistons. Thus, the fluid filling up the variable fluid volume space between the first 340 and second 350 pistons is forced to exit the chamber 310 through the outlet opening 330. The fluid is thus delivered by the dosing apparatus 100. From the pumping action, there are more than one option for the next state in the delivery cycle, wherein two of those options are shown in the coming figures.

FIG. 3D depicts a fourth state of the delivery cycle for a fluid, herein called the “reset state”. In this state, the first 231 and second 232 cam drives actuates a rotational movement of the first 211 and second 212 cam members in an opposite direction than to deliver the fluid, as in the previous states (direction illustrated by arrows R3 in the figure). The first 340 and second 350 pistons thus move laterally in the opposite direction along the axis A of the chamber 310 than to delivery the fluid. The first 340 and second 350 pistons return to an starting position, wherein the intake opening 320 and the outlet opening 330 are covered by the first 340 and/or second 350 pistons from within the chamber 310. From this reset state, the delivery cycle may restart a next cycle to deliver a fluid, and move to the suction state as illustrated in FIG. 3A. The first 211 and second 212 cam members thus rotate in a reciprocating motion together with the first 340 and second 350 pistons to deliver the fluid.

From the pumping action illustrated in FIG. 3C, a possible next state is shown in FIG. 3E, herein called the “switching state”. Instead of the first 231 and second 232 cam drives actuating a rotational movement of the first 211 and second 212 cam members in an opposite direction, the first 231 and second 232 cam drives may actuate a rotation in the same direction as when delivering a fluid (illustrated by an arrow R4 in the figure). The rotation of the first 211 and second 212 cam members is synchronized, such that the rotation happens simultaneously. The first 340 and second 350 pistons thus moves simultaneously instead of relative to each other. Since the first 221 and second 222 cam profile segments extend along a portion of the first 211 and second 212 cam members, a continuous rotation of the cam members 211, 212 may cause the first 360 and second 370 cam follower to be disengaged with the first 221 and second 222 cam profile segments. As is the case illustrated in FIG. 3E. The dosing delivery device 300 is disengaged from the dosing actuating unit 200 upon rotation of the first 211 and second 212 cam members. From this switching state, the dosing actuating unit 200 may engage with the next dosing delivery device 300 in the predefined sequence. When the following dosing delivery device 300 has been engaged, the delivery cycle may restart to delivery the next fluid. As shown in FIG. 1A or 1C, there are four dosing delivery devices 300, 300′, 300″, 300′″ arranged on the dosing actuating unit 200 simultaneously. The dosing apparatus 100 may for example switch between these four dosing delivery devices 300, 300′, 300″, 300′″ in a sequence, continuously cycling between delivering each of the fluids at a time.

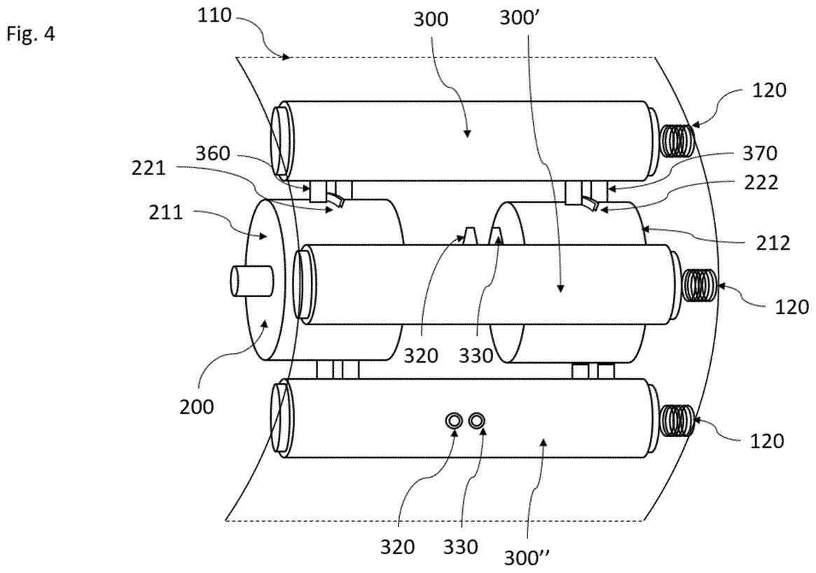

FIG. 4 illustrates a dosing apparatus comprising a frame 110 and an indexing member 120 according to an embodiment of the present disclosure. A portion of an embodiment of a frame 110 is shown, to illustrate an example for how a frame 110 may be arranged in relation to the dosing apparatus 100 and an indexing member 120. The frame 110 is arranged to cover the dosing apparatus 100. The frame 110 may thus protect the dosing apparatus 100 during use, or during transportation. The frame 110 may be made of a transparent material. In the illustrative example shown in this figure, there are three dosing delivery devices 300, 300′, 300″ arranged around the dosing actuating unit 200. Further, arranged on the frame 110 there are three indexing members 120. The indexing members 120 are compression springs. The indexing members 120 are fixed in one end to the frame 110. The other end of the indexing member is releasably engaged with the dosing delivery devices 300, 300′, 300″. The indexing members 120 hold the dosing delivery devices 300, 300′, 300″ in a desired position within the frame 110. According to an exemplifying embodiment illustrated in the figure, the desired position of the dosing delivery device 300 is to align the first 360 and second 370 cam follower with the first 221 and second 222 cam profile segments. Thus, when the first 231 and second 232 cam drives actuates a rotational movement of the first 211 and/or second 212 cam members to start a delivery of the fluid, the dosing delivery device 300 is in a position ready to engage with the dosing actuating unit 200. The dosing delivery devices 300 are spaced apart around the dosing actuating unit 200 such that they can engage with the first 221 and second 222 cam profile segments at different positions. The indexing member 120 is in a loaded state when engaged with the dosing delivery device 300, as illustrated in FIG. 4.

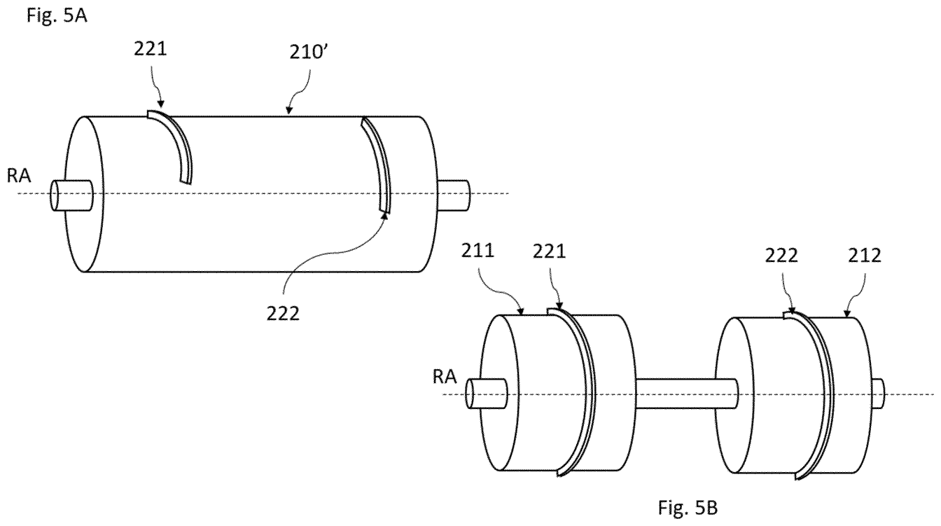

FIGS. 5A-5B illustrate possible embodiments of the dosing actuating unit 100.

FIG. 5A shows the dosing actuating unit comprises the cam element 210′ according to an embodiment of the present disclosure. The cam element 210′ is a barrel cam. The cam element 210 comprises a first cam profile segment 221, and a second cam profile segment 222 protruding from a surface of the cam element 210. The first 221 and second 222 cam profile segments extend helically around the cam element 210. The first 221 and second 222 cam profile segments extend around a portion of the circumference of the cam element 210′. Further, the first 221 and second 222 cam profile segments are arranged towards the opposite ends of the cam element 210′ along rotational axis RA. The first 221 and second 222 cam profile segments may cover different and/or overlapping portions of the circumference of the cam element 210′. In the embodiment shown in FIG. 5A, there may be only one drive unit 230 of the medicament delivery device (not shown in this figure) to actuate a rotational movement of the cam element 210.

FIG. 5B shows the dosing actuating unit comprises a first cam member 211 and a second cam member 212 arranged along rotational axis RA, and spaced apart from each other. In a preferred example, the first and the second cam members are barrel cams. The first cam member 211 comprises a first cam profile segment 221 extending around a portion or the entirety of the periphery of the first cam member 211. The first cam profile segment 221 extends helically around the first cam member 211. Further, the second cam member 212 comprises a second cam profile segment 222 extending around a portion or the entirety of the periphery of the second cam member 212. The second cam profile segment 222 extends helically around the second cam member 212. In the embodiment shown in FIG. 5B, there may be a first cam drive 231 actuating a rotational movement of the first cam member 211, and a second cam drive 232 actuating a rotational movement of the second cam member 212.

While the present disclosure is susceptible to various modifications and alternative forms, specific embodiments are shown and described by way of example in relation to the drawings, with a view to clearly explaining the various advantageous aspects of the present disclosure. It should be understood, however, that the detailed description herein and the drawings attached hereto are not intended to limit the disclosure to the particular form disclosed. Rather, the intention is to cover all modifications, equivalents, and alternatives falling within the scope of the following claims.

Some other aspects of the invention are defined by the following clauses.
1. A dosing apparatus (100) for parenteral delivery of at least two fluids, the dosing apparatus comprising:
a dosing actuating unit (200) comprising: a cam element (210; 210′) rotatably arranged around a rotational axis (RA); and a cam profile (221, 222) extending from the cam element (210; 210′);
wherein the cam element (210; 210′) is configured be connected to a drive unit and be rotated by the drive unit around the rotational axis (RA);
at least two dosing delivery devices (300, 300′, 300″, 300′″), each dosing delivery device comprising: a chamber (310) extending along an axis (A) and a first piston (340) movably arranged along the axis (A) within the chamber (310); wherein the chamber (310) is configured to accommodate a fluid; wherein the chamber (310) comprises an outlet opening (330) for deliver the accommodated fluid; wherein the first piston (340) comprises a first cam follower (360) configured to releasably engaged with the cam profile (221, 222) of the dosing actuating unit (200); wherein an interface formed between the cam profile (221, 222) and the first cam follower (360) is helical such that a rotational movement of the cam element is converted into an axial movement of the first piston along the axis (A) of the chamber (310).
2. The dosing apparatus according to clause 1, wherein each dosing delivery device of the at least two dosing delivery devices (300, 300′, 300″, 300′″) comprises a second piston (350) movably arranged along the axis (A) within the chamber (310); wherein the second piston (370) comprises a second cam follower (370) configured to releasably engaged with the cam profile (221, 222) of the dosing actuating unit (200).
3. The dosing apparatus according to clause 1 or 2, wherein the chamber (310) comprises an intake opening (320) open in a direction transverse to the axis (A); wherein the intake opening (320) is configured to be fluidly connected to a container containing a fluid; wherein the outlet opening is open in the direction transverse to the axis (A).
4. The dosing apparatus according to any of clauses 1-3, wherein the at least two dosing delivery devices are radially arranged around a circumference of the cam element.
5. The dosing apparatus according to any of preceding clauses, wherein the axis (A) of the chamber (310) of each dosing delivery device is parallel to the rotational axis (RA) of the cam element (210).
6. The dosing apparatus according to any one of the preceding clauses, wherein the cam profile is at least partially encircling a circumference of the cam element.
7. The dosing apparatus according to any of the preceding clauses, wherein the first cam follower is a rib protruding from the first piston in a direction transverse to the axis (A) of the chamber (310).
8. The dosing apparatus according to any of the preceding clauses, wherein the chamber (310) comprises at least a first piston opening through which the first cam follower protrudes; and wherein the first piston opening is open in the direction transverse to the axis (A) of the chamber (310).
9. The dosing apparatus according to any one of the preceding clauses, wherein the cam element (210) comprises a first cam member (211) and a second cam member (212), and wherein said first and second cam members (211, 212) are spaced apart from each other along the rotational axis (RA) of the cam element (210).
10. The dosing apparatus according to any one of the preceding clauses, wherein the chambers of the at least two dosing devices (300, 300′, 300″, 300′″) have different dimension to one another.
11. A medicament delivery device comprises the dosing apparatus according to any of the preceding clauses, wherein the medicament delivery device comprises:
a drive unit operably connected to the cam element;
a control unit configured to control the drive unit to activate one of the at least two dosing delivery devices to selectively deliver the one of the at least two fluids or deliver the at least two fluids.
12. The medicament delivery device according to clause 11, comprising a sensor electrically connected to the control unit; wherein the sensor is configured to detect the position of the cam profile.
13. The medicament delivery device according to clause 12, wherein the sensor is arranged on the cam element or arranged within the drive unit.
14. The medicament delivery device according to clause 12 or 13, wherein the sensor is at least one of an optical sensor, a magnetic sensor, or an encoder strip.
15. The medicament delivery device according to any of clauses 12-14, wherein the control unit is configured to, based on information from the sensor regarding the position of the cam profile, control the drive unit such that the cam element is rotated and engaged with the dosing delivery device for the selected fluid.
16. The medicament delivery device according to any of clauses 11-14, wherein the control unit controls an operation of the drive unit such that the at least two fluids are delivered in a predefined sequence.
17. The medicament delivery device according to any of clauses 11-16, wherein the control unit is configured to, based on a trigger input regarding the need for an emergency medication fluid delivery, adapt an operation of the drive unit such that, regardless of the predefined sequence, the emergency medication fluid is delivered next in order.
18. The medicament delivery device according to any of clauses 11-17 when dependent on a combination of clauses 2-3, wherein the control unit is configured to control the drive unit to rotate the cam element, such that
upon rotational movement of the cam element, the cam element actuates a movement of the first piston along the axis (A) of the chamber, causing a fluid to flow in through the one or more intake openings of the chamber, and
a movement of the second piston along the axis (A) of the chamber, causing the fluid to flow out through the one or more outlet openings of the chamber.
19. The medicament delivery device according to any of clauses 11-18, wherein the drive unit is a stepper motor.
20. The medicament delivery device according to any of clauses 11-19, comprising
an indexing member configured to be releasably engaged with a dosing delivery device at a predefined starting position, such that upon activation of a delivery of a fluid from the at least two fluids, the dosing actuation unit engages with the dosing delivery device containing the fluid in the starting position, whereby the dosing delivery device is released from the indexing member.
21. The medicament delivery device according to any of clauses 11-20, further comprising a frame configured to at least partly enclose the dosing actuating unit and the at least two dosing delivery devices.
22. The medicament delivery device according to a combination of clause 20 and 21, wherein the indexing member is fixed to the frame, and adjacent to one of the at least two delivery devices along the axis (A) of the chamber.
23. The medicament delivery device according to clause 22, wherein the indexing member is an elastic member.
24. The medicament delivery device according to clause 23, wherein the elastic member is a compression spring.
25. The medicament delivery device according to clause 20 or 22, wherein the indexing member is configured to break upon releasement of an engaged dosing delivery device, such that engagement with a further dosing delivery device is prevented.

1-15. (canceled)

16. A dosing apparatus for parenteral delivery of at least two fluids, the dosing apparatus comprising:
a dosing actuating unit comprising: a cam element rotatably arranged around a rotational axis;
a cam profile extending from the cam element, wherein the cam element is configured be connected to a drive unit and be rotated by the drive unit around the rotational axis; and
at least two dosing delivery devices, each dosing delivery device comprising: a chamber extending along an axis and a first piston movably arranged along the axis within the chamber, wherein the chamber is configured to accommodate a fluid, wherein the chamber comprises an outlet opening for deliver the accommodated fluid, wherein the first piston comprises a first cam follower configured to releasably engaged with the cam profile of the dosing actuating unit, and wherein an interface formed between the cam profile and the first cam follower is helical such that a rotational movement of the cam element is converted into an axial movement of the first piston along the axis of the chamber.

17. The dosing apparatus according to claim 16, wherein each dosing delivery device of the at least two dosing delivery devices comprises a second piston movably arranged along the axis within the chamber, and wherein the second piston comprises a second cam follower configured to releasably engage with the cam profile of the dosing actuating unit.

18. The dosing apparatus according to claim 16, wherein the chamber comprises an intake opening open in a direction transverse to the axis, wherein the intake opening is configured to be fluidly connected to a container containing a fluid, and wherein the outlet opening is open in the direction transverse to the axis.

19. The dosing apparatus according to claim 16, wherein the at least two dosing delivery devices are radially arranged around a circumference of the cam element.

20. The dosing apparatus according to claim 16, wherein the axis of the chamber of each dosing delivery device is parallel to the rotational axis of the cam element.

21. The dosing apparatus according to claim 16, wherein the first cam follower is a rib protruding from the first piston in a direction transverse to the axis of the chamber.

22. The dosing apparatus according to claim 16, wherein the chamber comprises at least a first piston opening through which the first cam follower protrudes; and
wherein the first piston opening is open in the direction transverse to the axis of the chamber.

23. The dosing apparatus according to claim 16, wherein the cam element comprises a first cam member and a second cam member, and wherein said first and second cam members are spaced apart from each other along the rotational axis of the cam element.

24. A medicament delivery device comprises the dosing apparatus according to claim 16, wherein the medicament delivery device comprises:
a drive unit operably connected to the cam element; and
a control unit configured to control the drive unit to activate one of the at least two dosing delivery devices to selectively deliver one of the at least two fluids or deliver the at least two fluids.

25. The medicament delivery device according claim 24, comprising a sensor electrically connected to the control unit, wherein the sensor is configured to detect the position of the cam profile.

26. The medicament delivery device according to claim 25, wherein the control unit is configured to, based on information from the sensor regarding the position of the cam profile, control the drive unit such that the cam element is rotated and engaged with the dosing delivery device for the selected fluid.

27. The medicament delivery device according to claim 24, wherein the control unit is configured to control the drive unit to rotate the cam element, such that
upon rotational movement of the cam element, the cam element actuates a movement of the first piston along the axis of the chamber, causing a fluid to flow in through the one or more intake openings of the chamber, and
a movement of the second piston along the axis of the chamber, causing the fluid to flow out through the one or more outlet openings of the chamber.

28. The medicament delivery device according to claim 24, further comprising:
an indexing member configured to be releasably engaged with a dosing delivery device at a predefined starting position, such that upon activation of a delivery of a fluid from the at least two fluids, the dosing actuation unit engages with the dosing delivery device containing the fluid in the starting position, whereby the dosing delivery device is released from the indexing member.

29. The medicament delivery device according to claim 24, further comprising:
a frame configured to at least partly enclose the dosing actuating unit and the at least two dosing delivery devices, wherein the indexing member is fixed to the frame, and adjacent to one of the at least two delivery devices along the axis of the chamber.

30. The medicament delivery device according to claim 28, wherein the indexing member is configured to break upon releasement of an engaged dosing delivery device, such that engagement with a further dosing delivery device is prevented.
